(19) United States
(12) Patent Application Publication
(10) Pub. No.: US 2005/0033353 A1
(43) Pub. Date: Feb. 10, 2005

(54) Apparatus for use in a percutaneous vasectomy

(75) Inventor: J. Stephen Jones, Shaker Heights, OH (US)

Correspondence Address: TROLLI, SUNDHEIM, COVELL & TUMMINO L.L.P.;SUITE 1111, 526 SUPERIOR AVENUE, CLEVELAND, OH, 44114-1400, US

(73) Assignee: The Cleveland Clinic Foundation

(21) Appl. No.: 10/898,797

(22) Filed: Jul. 26, 2004

Related U.S. Application Data
(60) Provisional application No. 60/490,864, filed on Jul. 29, 2003.

(51) Int. Cl. A61B 17/28

(52) U.S. Cl. 606/205

(57) ABSTRACT

An apparatus for use in a percutaneous vasectomy comprises first and second members pivotably connected to one another and movable between a closed condition and an open condition. Each of the first and second members has oppositely disposed first and second ends. Each of the first ends includes a handle portion. Each of the second ends includes an arcuate first portion and a second portion that has a semi-annular section. Each of the arcuate first portions terminates at a pointed tip for piercing through skin. The first portion of the first member and the first portion of the second member abut one another in the closed condition to form a unified tip. The semi-annular section of the second portion of the first member and the semi-annular section of the second portion of the second member abut one another in the closed condition to form a ring for encircling and isolating a structure.

### RELATED APPLICATION

This application claims the benefit of U.S. Provisional Application No. 60/490,864, which was filed on Jul. 29, 2003 and is incorporated herein by reference.

### TECHNICAL FIELD

The present invention relates to an apparatus for use in a percutaneous, “no-scalpel” vasectomy.

### BACKGROUND OF THE INVENTION

One method of birth control is vasectomy. Vasectomy is a surgical procedure for male sterilization that involves removal of at least a segment of the vas deferens. Vasectomy has traditionally been performed through one or two standard scrotal incisions. More recently, a “no-scalpel” vasectomy has also been developed. The no-scalpel approach is generally quicker and less-invasive than the traditional vasectomy, but can present certain challenges for the surgeon. For example, using the typical no-scalpel approach, the vas deferens is fixed against the inside of the skin on the scrotum with a ringed clamp. The scrotum is then pierced by a sharp-ended hemostat and the vas deferens is elevated through the skin by forceps for subsequent ligation of a segment of the vas. It can be tricky for the surgeon to perform the aforementioned piercing, elevating, and ligating steps, each of which utilize a different instrument, without losing control of the vas. Accordingly, an apparatus that frees the hand(s) of the surgeon during a percutaneous, no-scalpel vasectomy to enable the surgeon to perform two or more of the steps in the procedure with a single instrument is desirable.

### SUMMARY OF THE INVENTION

The present invention is an apparatus for use in a percutaneous medical procedure. The apparatus comprises first and second members pivotably connected to one another and movable between a closed condition and an open condition. Each of the first and second members has oppositely disposed first and second ends. Each of the first ends includes a handle portion. Each of the second ends includes an arcuate first portion and a second portion that has a semi-annular section. Each of the arcuate first portions terminates at a pointed tip for piercing through the skin. The first portion of the first member and the first portion of the second member abut one another in the closed condition to form a unified tip. The semi-annular section of the second portion of the first member and the semi-annular section of the second portion of the second member abut one another in the closed condition to form a ring for encircling and isolating a structure, such as a vessel.

In accordance with another feature of the invention, each of the first and second members includes a knife edge adjacent the second ends. The knife edges are engageable with one another to form scissors-like cutting means.

In accordance with another feature of the invention, each of the first ends of the first and second members includes a series of interlocking detents for locking the first and second members in the closed position.

In accordance with another feature of the invention, straight sections extend between and connect the first and second ends together.

In accordance with another feature of the invention, the arcuate first portions of the first and second members extend away from the straight sections of the first and second members at an obtuse angle.

In accordance with another feature of the invention, the semi-annular second portions of the first and second members extend away from the straight sections of the first and second members at an obtuse angle.

In accordance with another feature of the invention, the arcuate first portions of the first and second members extend at an angle away from the semi-annular second portions of the first and second members.

The present invention is also an apparatus for use in a percutaneous medical procedure. The apparatus comprises first and second members pivotably connected to one another and movable between a closed condition and an open condition. Each of the first and second members has oppositely disposed first and second ends connected together by straight sections. Each of the first ends includes a handle portion. Each of the second ends includes a portion that has a semi-annular section. The semi-annular section of the portion of the first member and the semi-annular section of the portion of the second member extend away from the straight section of the first and second members at an obtuse angle and the semi-annular sections abut one another in the closed condition to form a ring for encircling and isolating a structure.

The present invention is also an apparatus for use in a percutaneous “no-scalpel” vasectomy. The apparatus comprises first and second members pivotably connected to one another and movable between a closed condition and an open condition. Each of the first and second members has oppositely disposed first and second ends. Each of the first ends includes a handle portion. Each of the second ends includes an arcuate first portion and a second portion that has a semi-annular section. Each of the arcuate first portions terminates at a pointed tip for piercing through scrotal skin. The first portion of the first member and the first portion of the second member abut one another in the closed condition to form a unified tip. The semi-annular section of the second portion of the first member and the semi-annular section of the second portion of the second member abut one another in the closed condition to form a ring for encircling and isolating a vas deferens.

In accordance with another feature of the invention, each of the first and second members includes a knife edge adjacent the second ends. The knife edges are engageable with one another to form scissor-like cutting means for cutting the vas deferens.

The present invention is also a method for performing a percutaneous “no-scalpel” vasectomy. The method uses an apparatus which comprises first and second members pivotably connected to one another and movable between a closed condition and an open condition. Each of the first and second members has oppositely disposed first and second ends. Each of the first ends includes a handle portion. Each of the second ends includes an arcuate first portion and a second portion that has a semi-annular section. Each of the arcuate first portions terminates at a pointed tip for piercing through scrotal skin. The first portion of the first member and the first portion of the second member abut one another in the closed condition to form a unified pointed tip. The semi-annular section of the second portion of the first member and the semi-annular section of the second portion of the second member abut one another in the closed condition to form a ring for encircling and isolating a vas deferens. The method includes the steps of providing the apparatus, isolating a vas deferens; injecting anesthetic into the vasal sheath of the vas deferens; piercing the scrotal skin with the pointed tip of the apparatus; spreading the tip of the apparatus to the open condition under the pierced scrotal skin to expose the vas deferens; encircling the vas deferens with the first and second semi-annular sections of the apparatus by moving the first and second members to the closed condition around the vas deferens; pulling the vas deferens through the skin; and cutting the vas deferens.

In accordance with another feature of the method, after the step of spreading the tip, further including the steps of removing the tip of the apparatus from the spread incision; rotating the tip of the apparatus; and reinserting the first and second semi-annular sections of the apparatus into the incision.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of an apparatus for use in a percutaneous vasectomy, the apparatus being shown in an open position;

FIG. 2 is a perspective view of a portion of the apparatus of FIG. 1 and illustrating the apparatus in a closed position;

FIG. 3 is a side view of a portion of the apparatus of FIG. 2;

FIG. 4 is a view taken along line 4-4 in FIG. 3;

FIG. 5 is a perspective view of the apparatus of FIG. 1 during an initial step in a vasectomy;

FIG. 6 is a view similar to FIG. 5 illustrating a subsequent step in the vasectomy;

FIG. 7 is a view similar to FIG. 6 illustrating a another subsequent step in the vasectomy;

FIG. 8 is a view similar to FIG. 7 illustrating a yet another subsequent step in the vasectomy; and

FIG. 9 is a perspective view of a portion of an apparatus for use in a percutaneous vasectomy in accordance with a second embodiment of the present invention.

### DESCRIPTION OF EMBODIMENTS

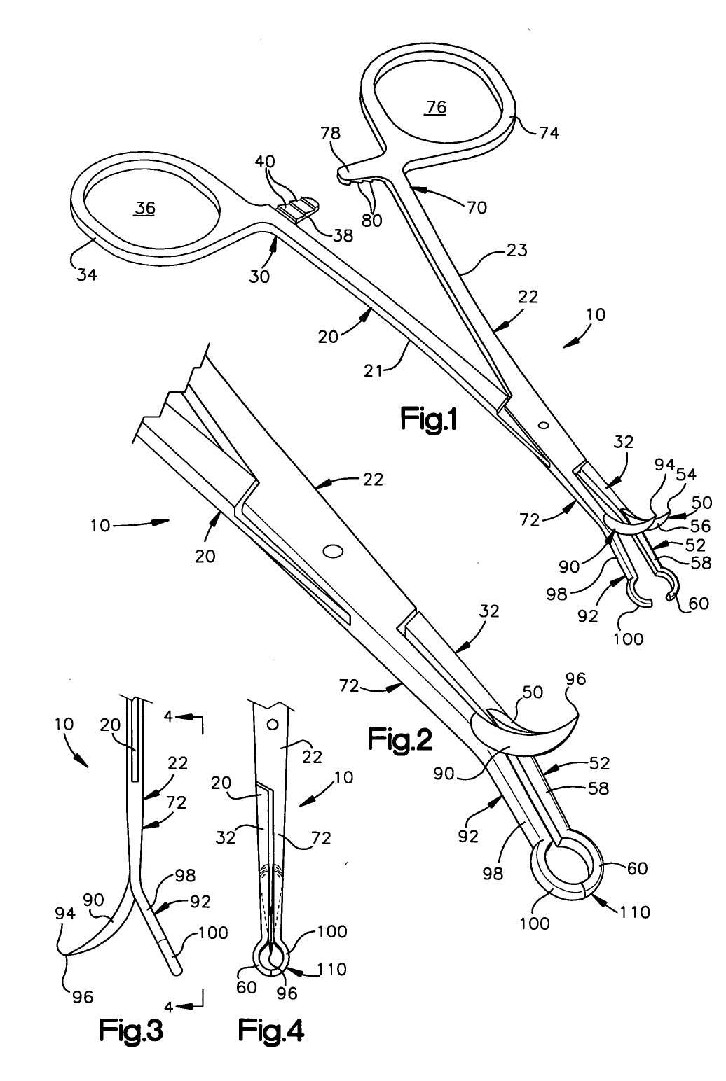

The present invention relates to an apparatus for use in a percutaneous, “no-scalpel” vasectomy. As representative of the present invention, FIG. 1 illustrates an apparatus 10 for use in a no-scalpel vasectomy. The apparatus 10 resembles a hemostat and comprises first and second members 20 and 22 that are mirror images of each other. The first and second members 20 and 22 are pivotably connected to one another and are movable between a closed condition (FIG. 2) and an open condition (FIG. 1). The first and second members 20 and 22 may be made from a medical grade metal so that the apparatus 10 is sterilizable and reusable. It is contemplated, however, that at least a portion of the apparatus 10 may be made from a rigid, medical grade polymer, with the portion(s) being replaced after each use.

The first member 20 has oppositely disposed first and second ends 30 and 32. A straight section 21 connects the first and second ends 30 and 32 of the first member 20 together. The first end 30 includes a handle portion 34 having an opening 36 for receiving a thumb or finger. The first end 30 further includes a projection 38 with a series of detents 40.

The second end 32 of the first member 20 includes an arcuate first portion 50 and a second portion 52. The arcuate first portion 50 curves away from the second portion 52, as may be seen in FIG. 3, and terminates at a pointed tip 54. The arcuate first portion 50 forms an obtuse angle with the straight section 21. The arcuate first portion 50 includes an inwardly facing planar surface 56 for abutting a similar surface on the second member 22.

The second portion 52 of the second end 32 projects away from the arcuate first portion 50 at an angle. The second portion 52 includes a straight section 58 and a semi-annular section 60.

Likewise, the second member 22 has oppositely disposed first and second ends 70 and 72. A straight section 23 connects the first and second ends 70 and 72 of the second member 22 together. The first end 70 includes a handle portion 74 having an opening 76 for receiving a thumb or finger. The first end 70 further includes a projection 78 with a series of detents 80 for engaging the detents 40 on the projection 38 on the first member 20.

The second end 72 of the second member 22 includes an arcuate first portion 90 and a second portion 92. The arcuate first portion 90 curves away from the second portion 92 and terminates at a pointed tip 94. The arcuate first portion 90 forms an obtuse angle with the straight section 23. The arcuate first portion 92 includes an inwardly facing planar surface (not numbered) for abutting the inwardly facing surface 56 on the first member 20 to form a unified tip 96 (FIG. 2).

The second portion 92 of the second end 72 projects away from the arcuate first portion 90 at an angle as may be seen in FIG. 3. The second portion 92 includes a straight section 98 and a semi-annular section 100. The semi-annular sections 60 and 100 of the first and second members 20 and 22, respectively, abut one another in the closed condition to form a ring 110 (FIGS. 2 and 4) for encircling and isolating a structure as is described further below.

To use the apparatus 10 in a vasectomy, a tripod grasp (FIG. 5) is utilized to trap one of the vas deferens 120 between the first and second fingers. The vas is isolated by gently “kneading” all cord structures (not shown) away so the it is isolated between the thumb and fingers. Lidocaine anesthetic is injected through a needle (not shown) that is advanced through the skin into the vasal sheath (not shown). This dissects the vasal sheath and other cord structures away from the vas deferens 120. Significantly, multiple needle passes are avoided.

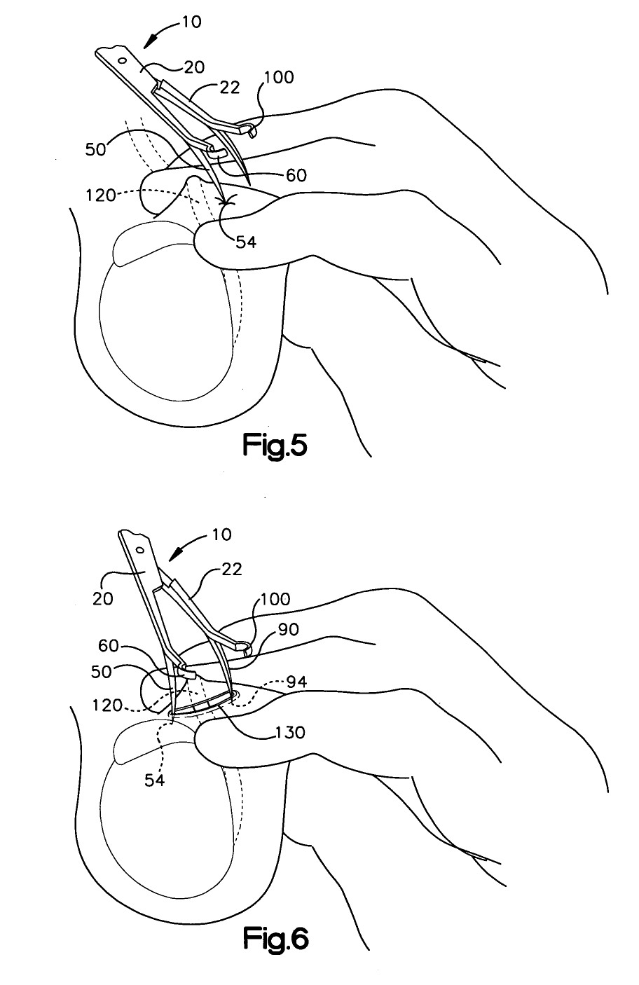

Instead of fixing the vas deferens 120 to the skin with a ringed clamp as is done in a typical no-scalpel vasectomy, the pointed tip 54 on the arcuate first section 50 of the first member 20 is pierced through the skin all the way to the vas deferens while holding the vas deferens securely in the tripod grasp. The pointed tips 54, 94 of the first portion of the first and second members 20 and 22 of the apparatus 10 are then moved to the closed condition, inserted into the incision 130 made by the tip 54, and spread to the open condition expose the vas deferens 120 as shown in FIG. 6.

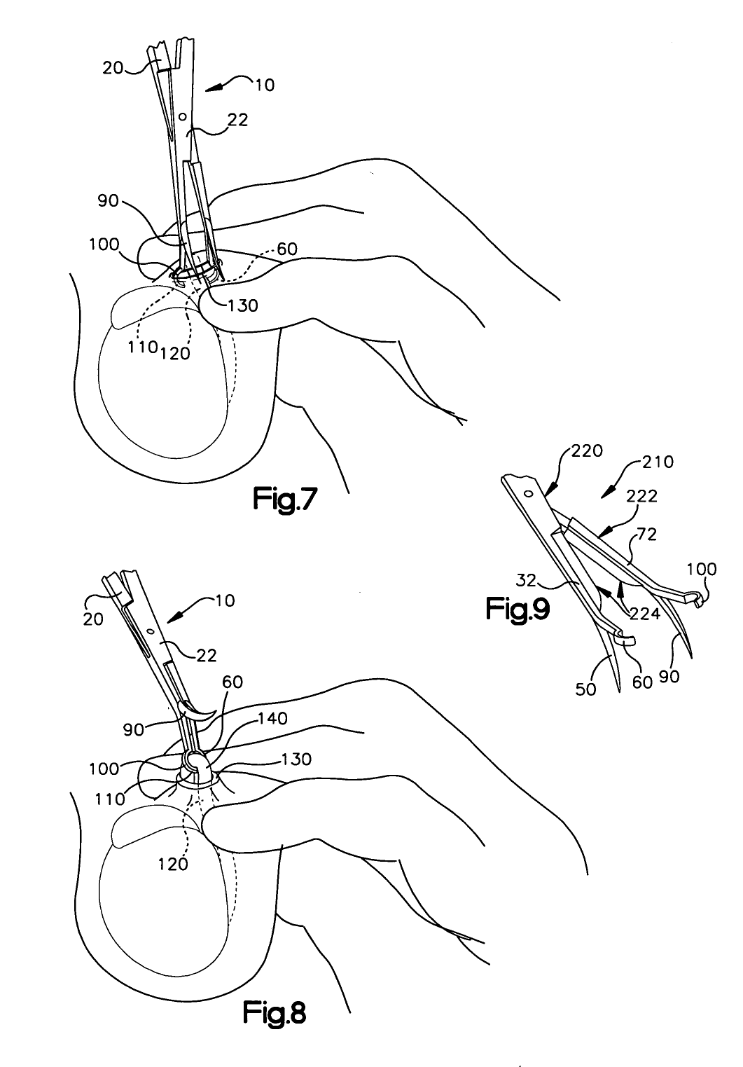

Subsequently, the first and second members 20, 22 of the apparatus 10 are removed from the spread incision and rotated. The semi-annular portions 60 and 100 are re-inserted into the incision (FIG. 7).

In the closed condition, the semi-annular sections 60 and 100 of the first and second members 20 and 22 that form the ring 110 are placed through the incision 130 and rolled over the vas deferens 120 to confirm location. The ring 110 is then opened beneath skin level and pressed firmly against the thumb to grasp the vas deferens 120 (FIG. 7). Until the surgeon is sure the vas deferens 120 is secured, the tripod grasp is maintained. Securing the vas deferens 120 with the ring 110 frees the physician's hand to handle other instruments during the procedure without losing control of the vas deferens. Once the vas deferens 120 is secured, the semi-annular sections 60 and 100 of the first and second members 20 and 22 are extracted to above skin level to pull a segment 140 of the vas deferens 120 through the incision 130 (FIG. 8).

The exposed aspect of the vasal sheath is incised with a scalpel (not shown) longitudinally. Cutting slightly into the vas wall assures the sheath is opened completely so the vas deferens 120 extrudes through the sheath. The vas deferens 120 can be re-grasped with another apparatus 10 as the severed sheath falls away exposing a 1-3 cm section of the vas deferens.

Following vasal excision and occlusion by the surgeon's method of choice, the procedure is repeated on the contralateral vas (not shown) from the opposite side of the table through a second incision. The procedure is repeated on the opposite vas after the surgeon moves to the other side of the bed.

The apparatus 10 described above thus provides a surgeon with the ability to perform two or more of the steps in a percutaneous, no-scalpel vasectomy with a single instrument to help prevent loss of control of the vas deferens 120. It should be understood, however, that the apparatus 10 described above could also be used to perform a typical no-scalpel vasectomy as described in the Background of the Invention.

FIG. 9 illustrates an apparatus 210 for use in a percutaneous vasectomy in accordance with a second embodiment of the present invention. In FIG. 9, reference numbers that are the same as reference numbers in the first embodiment of FIGS. 1-4 designate parts that are the same as parts in the first embodiment.

According to the second embodiment of FIG. 9, the apparatus 210 comprises first and second members 220 and 222 that are identical to the first and second members 20 and 22 except that each of the first and second members 220 and 222 includes a knife edge 224 adjacent their respective second ends 32 and 72. The knife edges 224 are engageable with one another to form a scissors-like cutting device for severing the vas deferens 120 once it is exposed and isolated.

From the above description of the invention, those skilled in the art will perceive improvements, changes and modifications. For example, it should be understood that the apparatus described above could be used in a variety of percutaneous medical procedures besides vasectomy in which a structure (vessel, object or otherwise) is to be isolated. For example, another method of birth control besides vasectomy is controlling female ovulation with hormones. The hormones can be taken orally on a daily basis, injected into the bloodstream or may be time-released into the body via an implant. Implants are an advantageous birth control method because after implantation into the body, the implants automatically slowly release hormones into the bloodstream over a long period of time, typically years, without the need for a patient to remember to take daily hormones.

One commonly used implant for slowly releasing hormones into the body over a period of five years for birth control is Norplant™. The Norplant™ implant comprises a series of five small stick-like structures implanted subcutaneously into the arm. When a patient decides she wants to become pregnant, the Norplant™ implant is simply removed in series using a percutaneous medical procedure. The apparatus 10 may be used to remove such an implant in a similar manner as when encircling and isolating the vas deferens as described in the vasectomy. It is further contemplated that the apparatus could include removable tip protectors made of a suitable material to protect both the surgical staff from an accidental stick and the tips from possible breakage. Such improvements, changes and modifications within the skill of the art are intended to be covered by the appended claims.

1. An apparatus for use in a percutaneous medical procedure, said apparatus comprising:
first and second members pivotably connected to one another and movable between a closed condition and an open condition, each of said first and second members having oppositely disposed first and second ends, each of said first ends including a handle portion, each of said second ends including an arcuate first portion and a second portion that has a semi-annular section, each of the arcuate first portions terminates at a pointed tip for piercing through skin;
said first portion of said first member and said first portion of said second member abutting one another in said closed condition to form a unified tip;
said semi-annular section of said second portion of said first member and said semi-annular section of said second portion of said second member abutting one another in said closed condition to form a ring for encircling and isolating a structure.

2. The apparatus of claim 1 wherein each of said first and second members includes a knife edge adjacent said second ends, said knife edges being engageable with one another to form scissors-like cutting means for cutting the isolated structure.

3. The apparatus of claim 1 wherein each of said first ends of said first and second members includes a series of interlocking detents for locking the first and second members in the closed position.

4. The apparatus of claim 1 wherein straight sections extend between and connect said first and second ends together.

5. The apparatus of claim 1 wherein said arcuate first portions of said first and second members extend away from said straight sections of said first and second members at an obtuse angle.

6. The apparatus of claim 1 wherein said semi-annular second portions of said first and second members extend away from said straight sections of said first and second members at an obtuse angle.

7. The apparatus of claim 1 wherein said arcuate first portions of said first and second members extend at an angle away from said semi-annular second portions of said first and second members.

8. An apparatus for use in a percutaneous medical procedure, said apparatus comprising:
first and second members pivotably connected to one another and movable between a closed condition and an open condition, each of said first and second members having oppositely disposed first and second ends connected together by straight sections, each of said first ends including a handle portion, each of said second ends including a portion that has a semi-annular section,
said semi-annular section of said portion of said first member and said semi-annular section of said portion of said second member extending away from said straight section of said first and second members at an obtuse angle and said semi-annular sections abutting one another in said closed condition to form a ring for encircling and isolating a structure.

9. The apparatus of claim 8 wherein each of said first and second members includes a knife edge adjacent said second ends, said knife edges being engageable with one another to form scissors-like cutting means for cutting the isolated structure.

10. The apparatus of claim 8 wherein each of said first ends of said first and second members includes a series of interlocking detents for locking the first and second members in the closed position.

11. The apparatus of claim 8 wherein each of said second ends include an arcuate portion, each of the arcuate portions terminates at a pointed tip for piercing through skin.

12. The apparatus of claim 11 wherein said arcuate portion of said first member and said arcuate portion of said second member abut each other in said closed condition to form a unified tip.

13. The apparatus of claim 12 wherein said arcuate portions of said first and second members extend away from said straight sections of said first and second members at an obtuse angle.

14. The apparatus of claim 1 wherein said semi-annular second portions of said first and second members extend away from said straight sections of said first and second members at an obtuse angle.

15. An apparatus for use in a percutaneous “no-scalpel” vasectomy, said apparatus comprising:
first and second members pivotably connected to one another and movable between a closed condition and an open condition, each of said first and second members having oppositely disposed first and second ends, each of said first ends including a handle portion, each of said second ends including an arcuate first portion and a second portion that has a semi-annular section, each of the arcuate first portions terminating at a pointed tip for piercing through scrotal skin;
said first portion of said first member and said first portion of said second member abutting one another in said closed condition to form a unified tip;
said semi-annular section of said second portion of said first member and said semi-annular section of said second portion of said second member abutting one another in said closed condition to form a ring for encircling and isolating a vas deferens.

16. The apparatus of claim 15 wherein each of said first and second members includes a knife edge adjacent said second ends, said knife edges being engageable with one another to form scissor-like cutting means for cutting the vas deferens.

17. The apparatus of claim 15 wherein each of said first ends of said first and second members includes a series of interlocking detents for locking the first and second members in the closed position.

18. The apparatus of claim 15 wherein straight sections extend between and connect said first and second ends together.

19. The apparatus of claim 15 wherein said arcuate first portions of said first and second members extend away from said straight sections of said first and second members at an obtuse angle.

20. The apparatus of claim 15 wherein said semi-annular second portions of said first and second members extend away from said straight sections of said first and second members at an obtuse angle.

21. The apparatus of claim 15 wherein said arcuate first portions of said first and second members extend at an angle away from said semi-annular second portions of said first and second members.

22. A method for performing a percutaneous “no-scalpel” vasectomy, said method comprising the steps of:
providing an apparatus comprising:
first and second members pivotably connected to one another and movable between a closed condition and an open condition, each of the first and second members having oppositely disposed first and second ends, each of the first ends including a handle portion, each of the second ends including an arcuate first portion and a second portion that has a semi-annular section, each of the arcuate first portions terminates at a pointed tip for piercing through scrotal skin;
the first portion of the first member and the first portion of the second member abutting one another in the closed condition to form a unified pointed tip;
the semi-annular section of the second portion of the first member and the semi-annular section of the second portion of the second member abutting one another in the closed condition to form a ring for encircling and isolating a vas deferens,
isolating a vas deferens;
injecting anesthetic into the vasal sheath of the vas deferens;
piercing the scrotal skin with the pointed tip of the apparatus;
spreading the tip of the apparatus to the open condition under the pierced scrotal skin to expose the vas deferens;
encircling the vas deferens with the first and second semi-annular sections of the apparatus by moving the first and second members to the closed condition around the vas deferens;
pulling the vas deferens through the skin; and
cutting the vas deferens.

23. The method according to claim 22 wherein after said step of spreading the tip, further including the steps of:
removing the tip of the apparatus from the spread incision;
rotating the tip of the apparatus; and
reinserting the first and second semi-annular sections of the apparatus into the incision.
